(19) United States
(12) Patent Application Publication
(10) Pub. No.: US 2015/0202237 A1
(43) Pub. Date: Jul. 23, 2015

(54) HALOBACTERIA EXTRACTS COMPOSITION FOR TUMOR REDUCTION

(71) Applicant: DR. NONA INTERNATIONAL LTD, Kanot (IL)

(72) Inventor: Nona KUCHINA, Rishon Lezion (IL)

(21) Appl. No.: 14/666,823

(22) Filed: Mar. 24, 2015

Related U.S. Application Data
(63) Continuation-in-part of application No. PCT/IL2013/050785, filed on Sep. 16, 2013.

(30) Foreign Application Priority Data
Sep. 24, 2012 (IL) 222128

(51) Int. Cl. A61K 35/74; A61K 36/05

(52) U.S. Cl. CPC A61K 35/74; A61K 36/05

(57) ABSTRACT

A composition and method for treating solid tumors in a mammalian subject. The composition includes halobacterial extracts and Dunaliella extracts, containing antioxidants. The composition also includes: (a) at least one water soluble fraction and, (b) at least one oil soluble fraction. The aforementioned extracts, together as a composition, are adapted for therapeutic treatment of solid tumor reduction. Furthermore, the composition is adapted to promote solid tumor reduction in radiation treatment in vitro and in vivo.

### FIELD OF THE INVENTION

This invention is directed towards a composition for treating tumor cells using a combination of halobacteria extracts and Dunaliella extracts. More specifically the invention relates to a composition comprising halobacteria and Dunaliella for enhancing radiation treatment influence and in parallel decreasing tumor cells in vitro and in vivo.

### BACKGROUND OF THE INVENTION

Halobacteria are known as halophilic microorganisms. This type of archaeon can act as a good model for some aspects of eukaryotic biology, such as DNA replication, transcription, and translation. Comparing a halophile genome to that of other prokaryotes should give insight into microbial adaptation to extreme conditions.

Halobacteria are extreme obligate bacteria. They require, for their growth, very high salt concentrations (from 10 to 30%), KCl, MgCl2 and especially NaCl. These organisms are isolated from natural media. To maintain their internal osmotic pressure which should be in equilibrium with the NaCl concentration in the medium, halobacteria accumulate from 3 to 4 M of salt in their cytoplasm in the form of KCl. A suspension of halobacteria in a medium containing an NaCl concentration of 2M causes complete loss of the stiffness of the bacterial envelope and the bacterium then assumes a round shape. Decreasing the salt concentration below 1 M leads to bacterial lysis.

Colonies of halobacteria are red in colour, their envelopes indeed contain coloured pigments (bacterio-ruberins) which protect them against intense ultraviolet radiation to which they are exposed. Halobacteria possess a pigment, halorhodopsin, which pumps chloride ions in the cell in response to photons, creating a voltage gradient and assisting in the production of energy from light. The process is unrelated to other forms of photosynthesis involving electron transport however, and halobacteria are incapable of fixing carbon from carbon dioxide.

The conventional shape of Halobacterium in a salt-rich medium is that of an oblong bacillus 4 to 10 [mu]m long and 0.7 [mu]m in diameter. This bacterium possesses from 5 to 8 lophotrichous flagella. Halobacterium halobium is incapable of using carbohydrates as carbon and energy source.

Variety of Halobacteria extracts are knows to have advantageous cosmetic properties especially for the treating scars burns or different types of sores as topical composition such as milk, cream, lotion, serum, mask or gel.

There thus remains an unmet and long felt need to provide means and method for facilitating treatment of proliferative diseases.

### SUMMARY OF THE INVENTION

It is one object of the present invention to provide a composition for treating solid tumors in a mammalian subject said composition comprising halobacterial extracts containing antioxidants comprising: (a)at least one water soluble fraction; and (b)at least one oil soluble fraction. The halobacterial extract is adapted for therapeutic treatment of solid tumor reduction; further wherein the halobacterial extract promotes solid tumor reduction in radiation treatment.

It is another object of the present invention to provide the composition as defined above, wherein the composition further comprises Dunaliella extracts.

It is one object of the present invention to provide an oral administrable tablet useful for treating irradiation and other related disorders, the tablet comprising: a halobacterial extract fraction, a Dunaliella extract fraction, at least one non-effervescent excipient or diluent, and at least one effervescent excipient.

It is one object of the present invention to provide a solid tumor reduction composition comprising halobacterial, wherein the composition is characterized by at least one measurable biological activity as determined by at least one bioassay, further wherein the composition has a anti tumorogenic activity fingerprint defined by Table 1.

It is one object of the present invention to provide a solid tumor reduction composition comprising halobacterial extract, wherein the composition acts as an anti-angiogenesis factor.

It is one object of the present invention to provide a solid tumor reduction composition comprising halobacterial extract, wherein the composition is characterized by interrupting the process of angiogenesis of solid tumor by facilitating a process selected from the group consisting of inhibiting proteases, inhibiting integrin signaling, inhibiting binding and activity of VEGF, affecting cell proliferation of endothelial cells, inhibiting bFGF, inhibiting cell migration, inducing apoptosis of endothelial cells, cell adhesion and survival of endothelial cells, antagonist of angiopoietin 1, or decoy receptors for VEGF-B and PIGF.

It is another object of the present invention to provide the composition as defined above, wherein the halobacteria extract is in an amount by weight of about 2.5%-10%

It is another object of the present invention to provide the composition as defined above, wherein the halobacterial extract comprises a therapeutic dose of halobacteria homogenate DN-1(halophilic archaea homogenate)

It is another object of the present invention to provide the composition as defined above, wherein the antioxidants composition confers a synergistic therapeutic radiosensitivity effect on subjects with respect to reduction of solid tumor.

It is another object of the present invention to provide the composition as defined above, wherein the synergistic effect is defined as at least 50% higher radiosensitivity to radiation treatment on cancer cells with respect to increasing the radiation damage of tumor cells in vitro.

It is another object of the present invention to provide the composition as defined above, wherein the synergistic effect is defined as a higher radiosensitivity to radiation treatment on subjects with respect to more pronounced regression of tumors.

It is another object of the present invention to provide the composition as defined above, wherein the synergistic effect is defined as an increscent of life span in comparison to a control group.

It is another object of the present invention to provide the composition as defined above, wherein the composition is administered orally.

It is another object of the present invention to provide the composition as defined above, wherein the orally administered composition form is selected from the group consisting of: tablets, capsules, caplets, or any oral suitable form.

It is another object of the present invention to provide the composition as defined above, wherein the Halobacteria extract, DN-1 Halobacteria homogenate has a modifying effect on radiosensitivity with gamma irradiation at 5 and 10 Gy.

It is another object of the present invention to provide the composition as defined above, wherein the Halobacteria extract, DN-1(I)—homogenate, prepared on 7% NaCl results a regression of about 10% tumor cells.

It is another object of the present invention to provide the composition as defined above, wherein the Halobacteria extract, DN-1 (I)—homogenate, prepared on 7% NaCl results less then 10% survival of tumor cells after irradiation of more than 16 Gy.

It is another object of the present invention to provide the composition as defined above, wherein the Halobacteria extract, DN-1 (II) homogenate, with the double concentration of Halobacteria prepared on 7% NaCl results a regression of about % tumor cells.

It is another object of the present invention to provide the composition as defined above, wherein the Halobacteria extract, DN-1 (II) homogenate, with the double concentration of Halobacteria prepared on 7% NaCl results less then 10% survival of tumor cells after irradiation of more then 10 Gy.

It is another object of the present invention to provide the composition as defined above, wherein the Halobacteria extract, DN-1(III)—homogenate, with the double concentration of Halobacteria prepared on 3.5% NaCl results a regression of 10% tumor cells.

It is another object of the present invention to provide the composition as defined above,wherein the Halobacteria extract, DN-1(III)—homogenate, with the double concentration of Halobacteria prepared on 3.5% NaCl results less then 10% a survival of tumer cells after irradiation of more then 10 Gy.

It is another object of the present invention to provide the composition as defined above, wherein the Halobacteria extract, DN-1 Halobacteria homogenate, effect upon the radiosensitivity of tumor cells irradiated in suspension results in pronounced regression of tumor cells.

It is another object of the present invention to provide the composition as defined above, wherein the Halobacteria extract, DN-1 Halobacteria homogenate, effect upon the radiosensitivity of tumor cells irradiated in suspension results in an increscent of a mammalian life span.

It is another object of the present invention to provide the composition as defined above, wherein the Halobacteria extract, halobacteria homogenate DN-1, increase the radiation damage of tumor cells in vitro and in vivo.

It is another object of the present invention to provide the composition as defined above, wherein the Halobacteria extract, halobacteria homogenate DN-1, reduces tumor cell proliferation.

It is another object of the present invention to provide the composition as defined above, wherein the Halobacteria extract, halobacteria homogenate DN-1, results the effect of suppression of malignant cell growth.

It is another object of the present invention to provide the composition as defined above, wherein the effect increases proportionally to DN-1 concentration and to the duration of treatment.

It is one object of the present invention to provide a method for extracting halobacteria homogenate DN-1, comprising the steps of: (a)providing halophilic bacteria, the bacteria being cultured in a medium which prevents rupture of the bacteria, (b)surrounding the bacteria in a medium comprising a bile salt and having a concentration of NaCl low enough to lyse the bacteria and cause the halobacteria homogenate DN-1 to be released into the medium, and (c) isolating the released halobacteria homogenate DN-1. The halobacteria homogenate DN-1 is adapted for therapeutic treatment of solid tumor reduction; further wherein the halobacteria homogenate DN-1 promotes solid tumor reduction in radiation treatment.

It is another object of the present invention to provide the method as defined above, wherein the medium in step (b) further comprises a detergent.

It is another object of the present invention to provide the method as defined above, wherein the medium in step (b) further comprises a cation complexing agent.

It is another object of the present invention to provide the method as defined above, wherein the detergent is selected from the group consisting of anionic detergents, cationic detergents, nonionic detergents, and amphoteric detergents.

It is another object of the present invention to provide the method as defined above, wherein the halobacteria extract is in an amount by weight of about 2.5%-10%.

It is another object of the present invention to provide the method as defined above, wherein the halobacterial extract comprises a therapeutic dose of halobacteria homogenate DN-1.

It is another object of the present invention to provide the method as defined above, wherein the antioxidants composition confers a synergistic therapeutic radio sensitivity effect on subjects with respect to reduction of solid tumor.

It is another object of the present invention to provide the method as defined above, wherein the synergistic effect is at least 50% higher radiosensitivity to radiation treatment on cancer cells with respect to increasing the radiation damage of tumor cells in vitro.

It is another object of the present invention to provide the method as defined above, wherein the synergistic effect is defined as a higher radiosensitivity to radiation treatment on subjects with respect to more pronounced regression of tumors.

It is another object of the present invention to provide the method as defined above, wherein the synergistic effect is defined as an increase of life span in comparison to a control group.

It is another object of the present invention to provide the method as defined above, wherein the composition is administered orally.

It is another object of the present invention to provide the method as defined above, wherein the orally administered composition is in the form selected from the group consisting of: tablets, capsules, caplets, or any oral suitable form.

It is another object of the present invention to provide the method as defined above, wherein the Halobacteria extract, halobacteria homogenate DN-1, has a modifying effect on radiosensitivity with gamma irradiation at 5 and 10 Gy.

It is another object of the present invention to provide the method as defined above, wherein the Halobacteria extract, halobacteria homogenate DN-1(I), prepared on 7% NaCl results a regression of about 10% tumor cells.

It is another object of the present invention to provide the method as defined above, wherein the Halobacteria extract, DN-1 (I)—homogenate, prepared on 7% NaCl results less then 10% survival of tumor cells after irradiation of more then 16 Gy.

It is another object of the present invention to provide the method as defined above, wherein the Halobacteria extract, DN-1 (II) homogenate, with the double concentration of Halobacteria prepared on 7% NaCl results a regression of about 10% tumor cells.

It is another object of the present invention to provide the method as defined above, wherein the Halobacteria extract, DN-1 (II) homogenate, with the double concentration of Halobacteria prepared on 7% NaCl results less then 10% survival of tumor cells after irradiation of more then 10 Gy.

It is another object of the present invention to provide the method as defined above, wherein the Halobacteria extract, DN-1(III)—homogenate, with the double concentration of Halobacteria prepared on 3.5% NaCl results a regression of 10% tumor cells.

It is another object of the present invention to provide the method as defined above, wherein the Halobacteria extract, DN-1(III)—homogenate, with the double concentration of Halobacteria prepared on 3.5% NaCl results less then 10% a survival of tumer cells after irradiation of more then 10 Gy.

It is another object of the present invention to provide the method as defined above, wherein the Halobacteria extract, DN-1 Halobacteria homogenate, effect upon the radiosensitivity of tumor cells irradiated in suspension results in pronounced regression of tumor cells.

It is another object of the present invention to provide the method as defined above, wherein the Halobacteria extract, DN-1 Halobacteria homogenate, effect upon the radiosensitivity of tumor cells irradiated in suspension results in an increase of a mammalian life span.

It is another object of the present invention to provide the method as defined above, wherein the Halobacteria extract, halobacteria homogenate DN-1, increases the radiation damage of tumor cells in vitro and in vivo.

It is another object of the present invention to provide the method as defined above, wherein the Halobacteria extract, halobacteria homogenate DN-1, reduces tumor cell proliferation.

It is another object of the present invention to provide the method as defined above, wherein the Halobacteria extract, halobacteria homogenate DN-1, results the effect of suppression of malignant cell growth.

It is another object of the present invention to provide the method as defined above, wherein the effect increases proportionally to DN-1 concentration and to the duration of treatment.

### BRIEF DESCRIPTION

In order to understand the invention and to see how it may be implemented in practice, a plurality of embodiments is adapted to now be described, by way of non-limiting example only, with reference to the accompanying drawings, wherein:

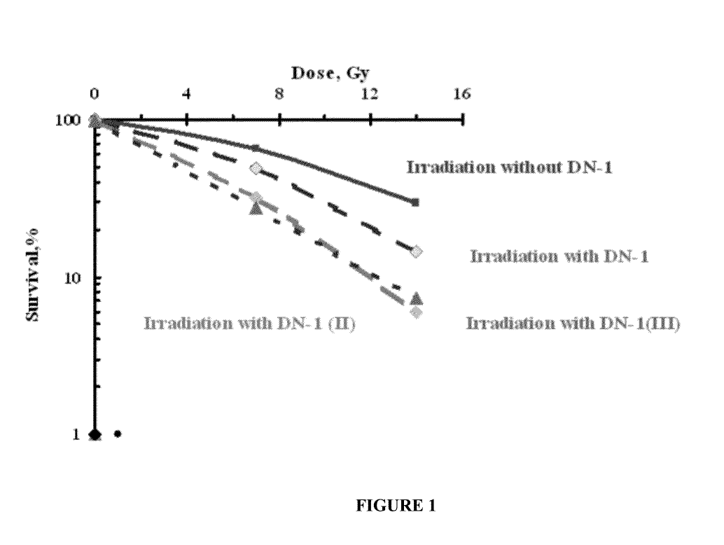

FIG. 1 presents a graph of survival of B-16 melanoma cells irradiated in vitro in suspension, in accordance with a preferred embodiment of the present invention;

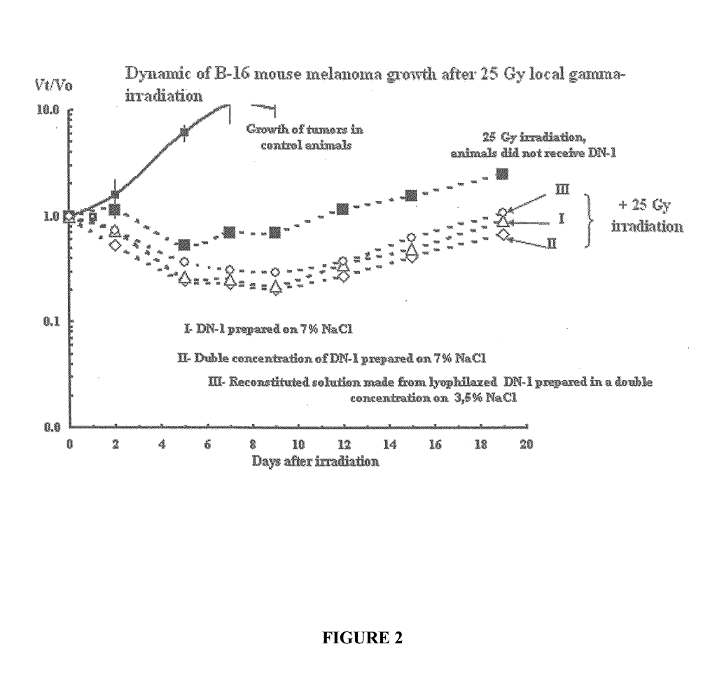

FIG. 2 presents a graph of dynamic of B-16 mouse melanoma growth after 25 Gy local gamma-irradiation, in accordance with a preferred embodiment of the present invention;

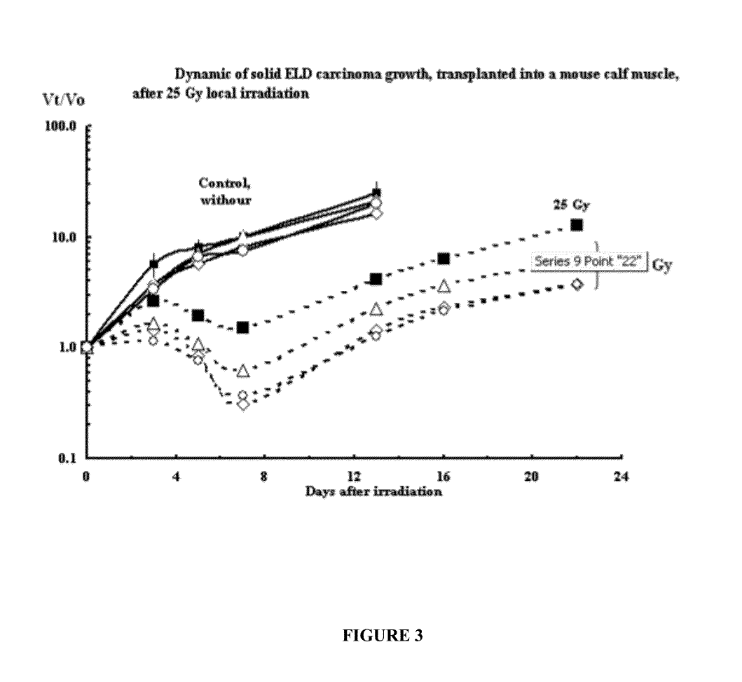

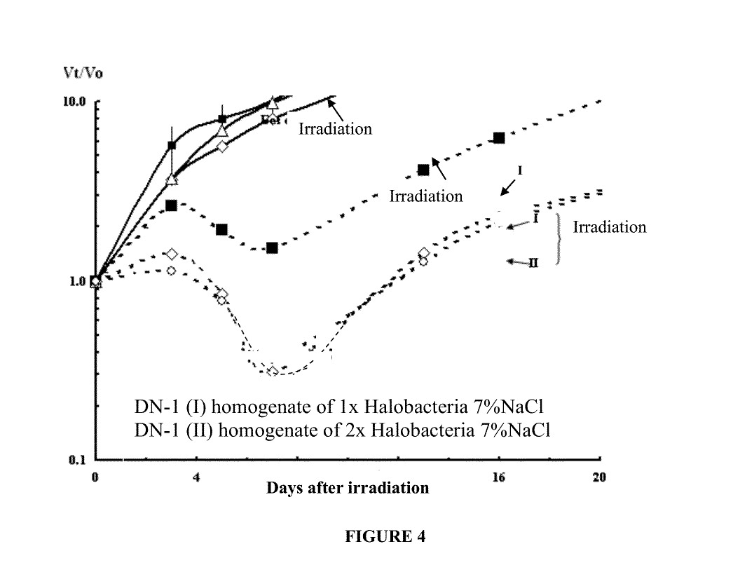

FIGS. 3-4 present a graph of dynamic of solid ELD carcinoma growth after 25 Gy local gamma-irradiation, in accordance with a preferred embodiment of the present invention;

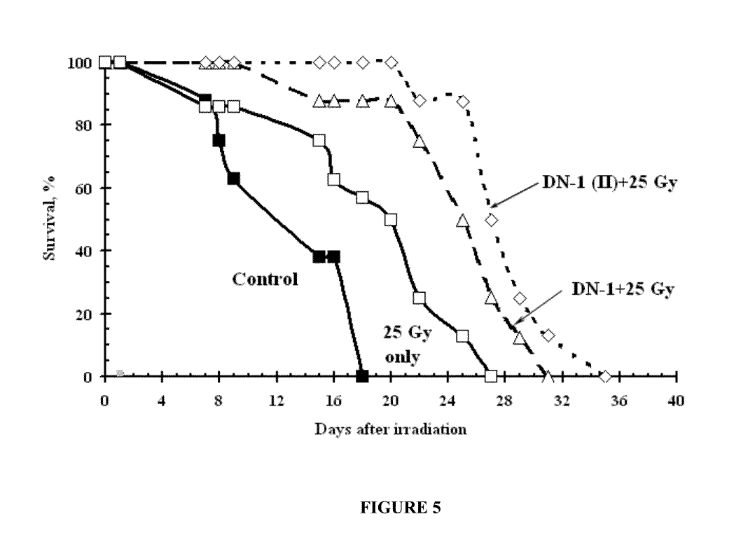

FIG. 5 presents a graph of survival of mice with B-16 melanoma after local gamma irradiation, in accordance with a preferred embodiment of the present invention;

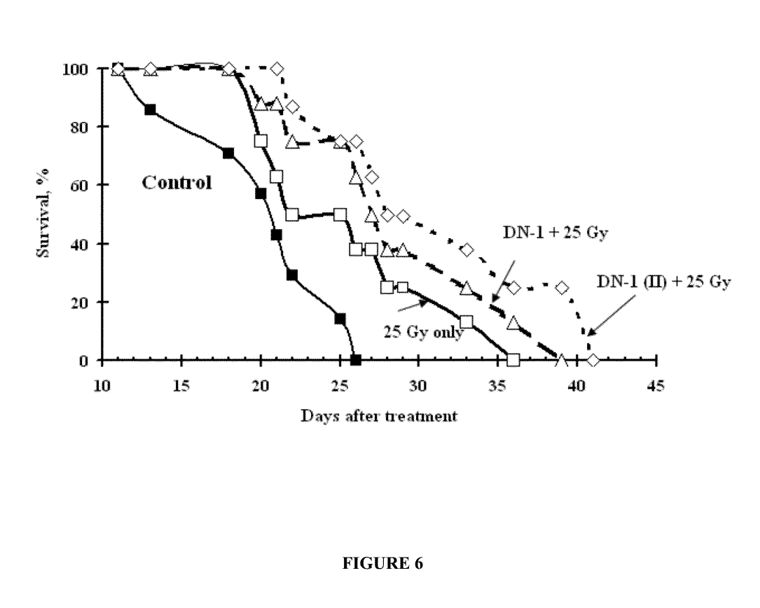

FIG. 6 presents a graph of survival of mice with ELD tumor after local gamma irradiation, in accordance with a preferred embodiment of the present invention;

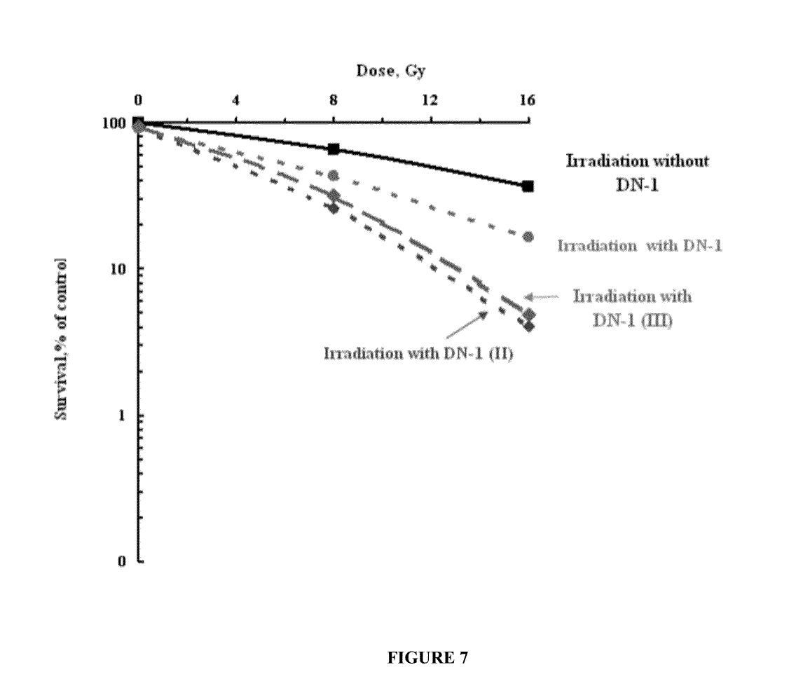

FIG. 7 presents a graph of survival of V-79 cells irradiated in vitro in suspension, in accordance with a preferred embodiment of the present invention;

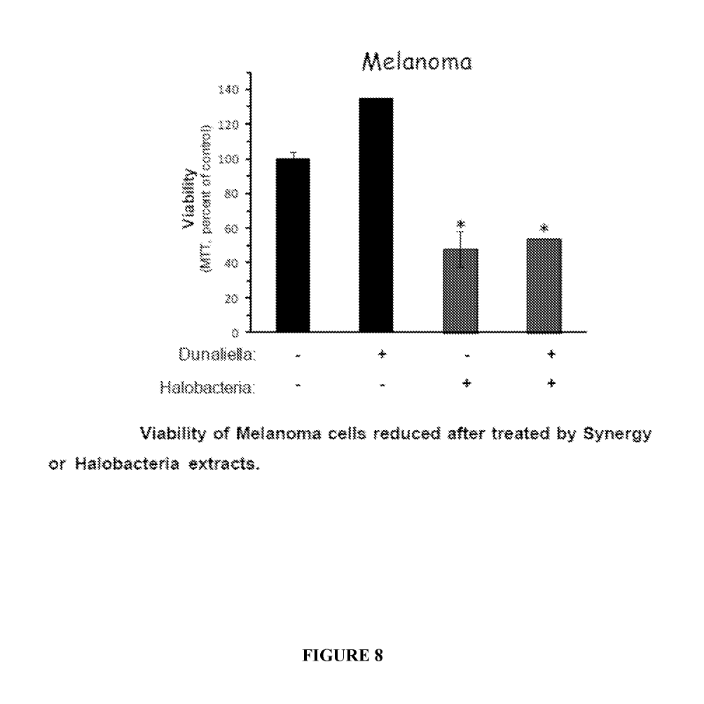

FIG. 8 presents a graph of reduction of viability of Melanoma cells after treated by Synergy or Halobacteria extracts, in accordance with a preferred embodiment of the present invention;

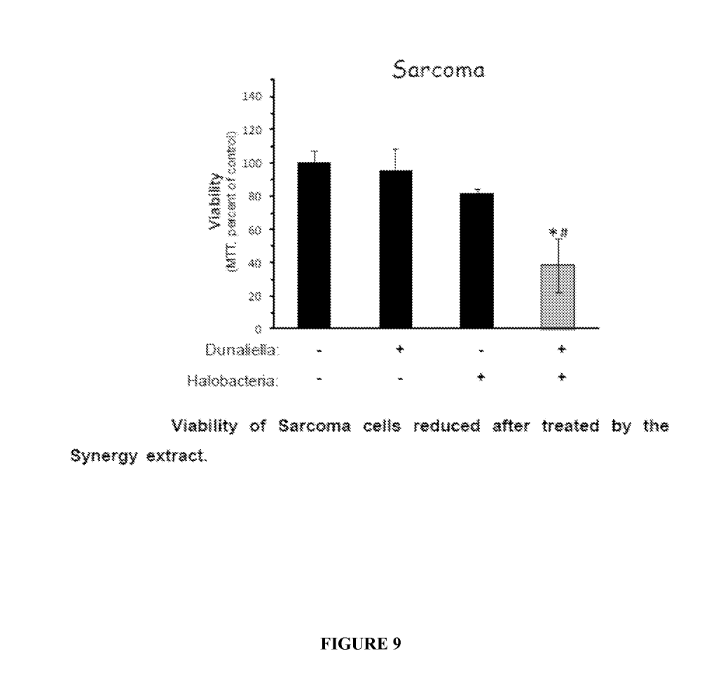

FIG. 9 presents a graph of reduction of viability of Sarcoma cells after treated by the Synergy extract, in accordance with a preferred embodiment of the present invention;

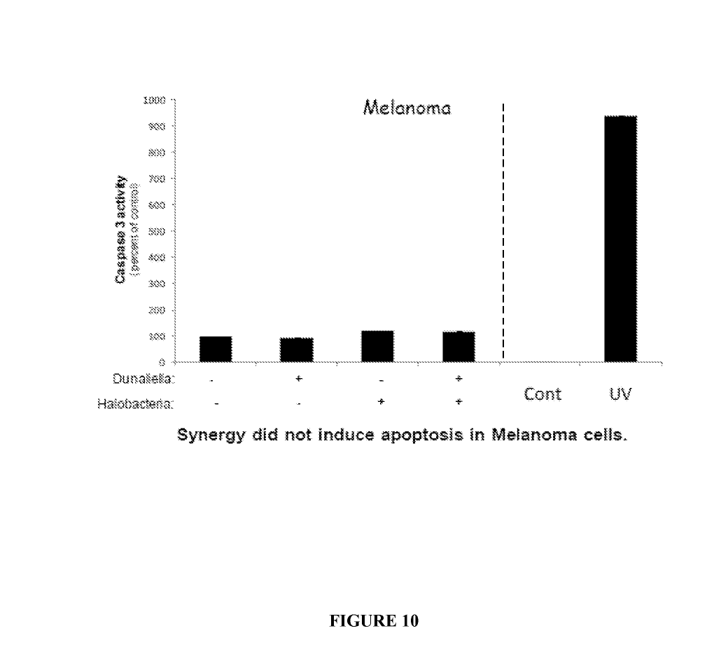

FIG. 10 presents a graph showing that the synergy compound did not induce apoptosis in Melanoma cells, in accordance with a preferred embodiment of the present invention;

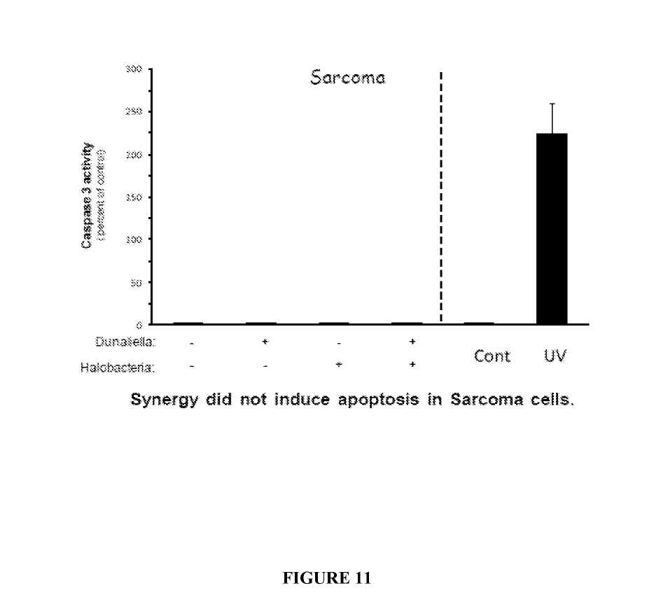

FIG. 11 presents a graph showing that the synergy compound did not induce apoptosis in Sarcoma cells, in accordance with a preferred embodiment of the present invention;

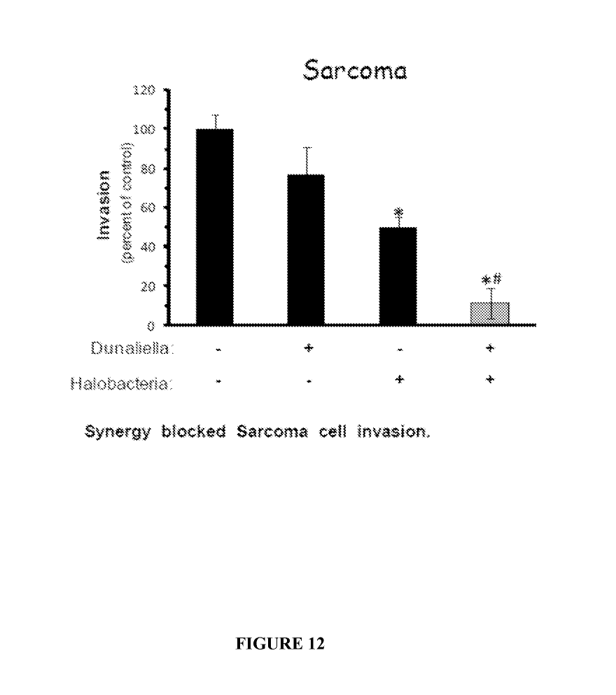

FIG. 12 presents a graph showing that the synergy compound blocked Sarcoma cell invasion, in accordance with a preferred embodiment of the present invention;

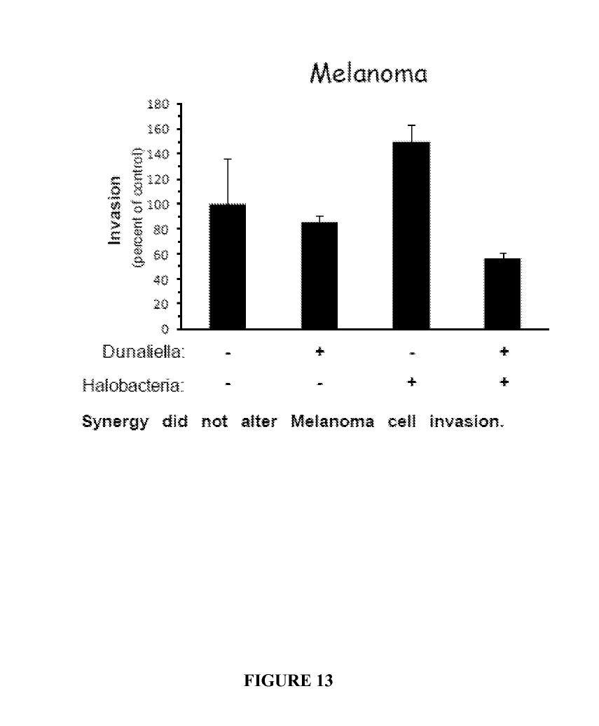

FIG. 13 presents a graph showing that the synergy compound did not alter Melanoma cell invasion, in accordance with a preferred embodiment of the present invention;

### DETAILED DESCRIPTION

The following description is provided so as to enable any person skilled in the art to make use of the invention and sets forth the best modes contemplated by the inventor of carrying out this invention. Various modifications, however, will remain apparent to those skilled in the art, since the generic principles of the present invention have been defined specifically to provide archaebacteria DN-1 and extracts useful for enhancing radiation treatment influence and in parallel decreasing tumor cells in vitro and in vivo. The present invention is a product which includes archaebacteria extract DN-1 which comprises strong antioxidants that dissolved in oil and in water. The product has further a wide range impact on rehabilitation of the skin. The archaebacteria extract DN-1 is delivered orally.

The term “Halobacteria”, “Archaebacteria”, “halophilic archaea halobacteria” as used herein should be further understood also as archaebacterium, Halobacterium halobium.

Halobacteria are recognized as archaea, rather than bacteria. The name ‘halobacteria’ was assigned to this group of organisms before the existence of the domain Archaea was realized, and remains valid according to taxonomic rules. In a non-taxonomic context, halophilic archaea are also sometimes referred to as haloarchaea to distinguish them from halophilic bacteria.

Halobacteria are unique in that they perform photosynthesis without chlorophyll. Instead, their photosynthetic pigments are bacteriorhodopsin and halorhodopsin. These pigments are similar to sensory rhodopsin, the pigment used by humans and other animals for vision. Bacteriorhodopsin and halorhodopsin are embedded in the cell membranes of halobacteria and each pigment consists of retinal, a vitamin-A derivative, bound to a protein. Irradiation of these pigments causes a structural change in their retinal. This is referred to as photoisomerization.

Retinal photoisomerization leads to the synthesis of ATP. Halobacteria have two additional rhodopsins, sensory rhodopsin-I and sensory rhodopsin-II. These compounds regulate phototaxis, the directional movement in response to light.

Halobacteria are extreme obligate bacteria. They indeed require, for their growth, very high salt concentrations (from 10 to 30%), KCl, MgCl2 and especially NaCl. These organisms have been isolated from natural media or artificial media (salterns). To maintain their internal osmotic pressure which should be in equilibrium with the NaCl concentration in the medium, halobacteria accumulate from 3 to 4 M of salt in their cytoplasm in the form of KCl. A suspension of halobacteria in a medium containing an NaCl concentration of 2M causes complete loss of the stiffness of the bacterial envelope and the bacterium then assumes a round shape. Decreasing the salt concentration below 1M leads to bacterial lysis.

Colonies of halobacteria are red in colour, their envelopes indeed contain coloured pigments (bacterio-ruberins) which protect them against intense ultraviolet radiation to which they are exposed.

Products that contain antioxidants have a positive effect on patients undergoing radiation therapy, and also help the patients in case of complications of radiotherapy (Mucositis).

DN-1 contains two groups of antioxidants—water soluble and oil soluble, so it is an antioxidant containing extract with wide-ranging effects on body restoration after radiation, wounds, burns, pressure sores and scarring after surgery.

The present invention further presents the ability of Halobacteria homogenate DN-1 to increase the radiation damage of solid tumor cells in vitro and in vivo.

Solid tumors may be benign or malignant. Examples of solid tumors are sarcomas, carcinomas, and lymphomas. Leukemias generally do not form solid tumors.

Sarcomas are cancers formed from the connective tissues in the body, such as bone or muscle. This type of solid tumor is assigned a grade of low, intermediate or high depending on the cellular characteristics of the cancer. Low-grade sarcomas are usually treated with surgery. Intermediate and high-grade sarcomas are usually treated with a combination of surgery, chemotherapy and radiation.

Carcinomas are born in the body's glandular and epithelial cells. These cells line the air passages and gastrointestinal tract in the body. Adrenocortical carcinomas arise in the adrenal cortex, which makes hormones that help the body work properly. Thyroid carcinomas arise from the thyroid, which produces hormones that affect heart rate, body temperature, energy level and calcium level. Other carcinomas include nasopharyngeal carcinoma, which affects the upper throat, and skin carcinoma.

Lymphomas arise in the lymphoid organs, which include the lymph nodes, spleen and thymus. These organs are responsible for producing and storing cells that help the body fight infection. Lymphomas are the most common form of blood cancer in the developed world and account for 5.3 percent of all cancers in the United States.

There are no clear symptoms for a solid tumor because of the wide variety of organs it can develop in. The symptoms a person feels depends on which organ the cancer has targeted, where in that organ the solid tumor is located, the rate of growth of cancer cells, and whether the cancer has spread to other organs (metastasis).

Treatments for solid tumors depend on a number of factors including the cancer's location, the stage, and the patient's health. Other options to remove the tumor, except surgery include chemotherapy and radiation therapy.

The dosage forms of the halobacteria composition of the present invention include pill, tablet, capsule, natural form or any suitable oral solid form. Notably, the route of administration (ROA) for drug delivery is dependent on the dosage form of the substance. The composition of the present invention may further comprises Dunaliella extracts.

Dunaliella salina is a type of halophile green micro-algae especially found in sea salt fields. Dunaliella is known for its anti-oxidant activity because of its ability to create large amount of caroteneoids.

The Dunaliella, a halotolerant green alga, accumulate high concentration of β-carotene when grown under defined condition. Dunaliella posses the ability to accumulate very large amounts of β-carotene (more than 10% of the algae dry weight) under defined condition. The extent of β-carotene accumulation was shown to have a direct function of the integral amount of light and high NaCl concentration to which the algae are exposed during a division cycle.

β-carotene possesses powerful anti-cancer properties. By reducing the amount of harmful free radicals in the body that can otherwise damage the DNA which further promotes cosmetic-related problems such as wrinkles and, on a more serious note, it can increase a subject risk of cancer. Dunaliella known to have a direct influence on the immune cells.

In another embodiment, the cell membrane of algae of the genus Dunaliella becomes hydrophobic when the algae are in contact with solutions of sodium chloride having a concentration of about 3M or higher, and this behavior enables the algae to be adsorbed on to substances having a hydrophobic surface, thus providing a means whereby the algae can be rapidly and economically separated and recovered from the saline medium in which they have been grown. It was found that at lower concentrations of sodium chloride such as that found in sea water the surface of the cell membrane is dominated by polar groups and is not hydrophobic and hence the algae will not adsorb or remain adsorbed on a hydrophobic surface. Dunaliella further contains carotenoid called zeaxanthin, a valuable antioxidant with the ability to both help prevent and treat debilitating condition that causes progressive vision loss.

The combination of Dunaliella and halobacteria further enhances the radiation treatment influence and in parallel decreasing tumor cells in vitro and in vivo.

The composition of the present invention defined above, wherein the antioxidants composition confers a synergistic therapeutic radiosensitivity effect on subjects with respect to reduction of solid tumor.

The composition of the present invention defined above, wherein the synergistic effect is defined as at least 50% higher radiosensitivity to radiation treatment on cancer cells with respect to increasing the radiation damage of tumor cells in vitro.

The composition of the present invention defined above, wherein the synergistic effect is defined as a higher radiosensitivity to radiation treatment on subjects with respect to more pronounced regression of tumors.

The composition of the present invention defined above, wherein the synergistic effect is defined as an increasment of life span in comparison to a control group.

The composition of the present invention defined above, wherein the composition is administered orally.

The composition of the present invention defined above, wherein the orally administered composition is in the form selected from the group consisting of: tablets, capsules, caplets, or any oral suitable form.

The composition of the present invention defined above, wherein the Halobacteria extract, DN-1 Halobacteria homogenate has a modifying effect on radiosensitivity with gamma irradiation at 5 and 10 Gy.

### EXAMPLES

FIGS. 1-7 present the effect of DN-1 Halobacteria homogenate on the radiosensitivity of tumor cells irradiated in suspension.

B-16 mouse melanoma cells and V-79 Chinese hamster were gamma-irradiated in suspension in vivo and in vitro.

The mice were divided to different group while one group tested without addition of DN-1 or 15 min after addition to suspension of 10% of DN-1 which were prepared with different concentration of Halobacteria cells or in different concentration of NaCl solutions: 3.5% NaCl concentration and 7% NaCl concentration. The ability of the cells to form the visible colonies, clonogenicity, was an end point and was considered as the survival of the cell.

The present procedure is applicable to the extraction of halobacteria. It is based on the weakness of the cell envelopes of these microorganisms when they are exposed to low concentrations of salts, for example, in fresh water; under these conditions the cells of halophilic bacteria lyse (rupture), releasing all the cell components into the medium.

The following examples are intended to illustrate the present invention without, however, being of a limiting nature:

In Vitro

The present invention discloses the effect of DN-1 preparation on proliferation of intact and irradiated malignant and normal cells in vitro. DN-1 is a homogenate halobacteria prepared on the 7.5% NaCl.

The effect of 0.1 3% DN-1 in the growth medium on the rate of cell division has been studied for mouse tumor cells: B-16 melanoma, Lewis lung carcinoma, and EMT-6 mammary carcinoma, as well as HT-29 human colon carcinoma and epithelial cells of fetal human colon. The modifying effect of DN-1 on radiosensitivity was assessed for several cell lines in experiments with gamma irradiation at 5 and 10 Gy.

The earlier data of several authors indicating suppression of malignant cell growth by DN-1 have been confirmed in two cell lines. This effect is increasing proportionally to DN-1 concentration and duration of treatment. However, EMT-6 cells are exception, and their rate of division was not influenced by DN-1 in the stated concentration.

Addition of 3% DN-1 into medium for 1 h before and during period of gamma irradiation increased radiation death of Lewis lung carcinoma cells, B-16 melanoma and HT-29 human colon carcinoma, but did not modify damage of epithelial cells of normal human intestine. DN-1 substantially inhibits proliferation of two mouse tumor cells in vitro: B-16 and Lewis lung carcinoma, but did not influence proliferation of EMT-6 cells. DN-1 administration into the growth medium for one hour before and during irradiation substantially increases radiation damage of malignant cells of human origin (HT-29) without change in sensitivity of normal cells of similar origin.

The effect of DN-1 Halobacteria homogenate was further presented on the radiosensitivity of tumor cells irradiated in suspension. B-16 mouse melanoma cells and V-79 Chinese hamster were gamma-irradiated in suspension without addition of DN-1 or 15 min after addition to suspension of 10% of DN-1 prepared with different concentration of Halobacteria cells or in NaCl solutions with the diminished (3.5% instead of 7% NaCl concentration). The ability of cells to form the visible colonies (clonogenicity) was an end point and is considered as cell “survival”.

Radiosensitivity is the relative susceptibility of cells, tissues, organs or organisms to the harmful effect of ionizing radiation. Cells are least sensitive when in the S phase, then the G₁ phase, then G₂ phase and the most sensitive in the M phase of the cell cycle. According to the law of Bergonié and Tribondeau quickly dividing tumor cells are generally more sensitive than the majority of body cells. This is not always true. Tumor cells can be hypoxic and therefore less sensitive to X-rays that mediate most of their effects through free radicals produced by ionizing oxygen. Later it has been shown that the most sensitive cells are those that are undifferentiated, well nourished, divide quickly and are highly metabolically active. Amongst the body cells, the most sensitive are spermatogonia and erythroblasts, epidermal stem cells, gastrointestinal stem cells. The least sensitive are nerve cells and muscle fibers. Very sensitive cells are also oocytes and lymphocytes, although they are resting cells and do not meet the criteria described above.

In Vivo

The effect of DN-1 Halobacteria homogenate was studied on the dynamic of regression of two tumors—ELD carcinoma and B-16 melanoma—transplanted in the shin muscles of mice after tumor gamma-irradiation and on the duration of mice survival after treatment.

Irradiation was done on the clinical Co machine supplied with the additional screen for the mice bodies. The irradiation was performed 6-7 days after tumor transplantation when the diameters of the shins increased from ˜4 to 8-9 mm.

The ‘experimental’ mice received daily per os 0.2 ml of diluted (1:10) solutions of DN-1 prepared with the usual 46 mg (variant I) or double, 96 mg (variant II) amount of Halobacteria cell mass in 1 ml of 7% NaCl as well as reconstituted from DN-1 prepared in 3.5% NaCl and then lyophilized (variant III).

The tumors volumes were measured after treatment three times a week. The results are presented on FIGS. 2-4 for tumors regressions and recurrence and on FIG. 1 and FIG. 5-7 for mice survival.

The ‘feeding’ of animals with DN-1 results in more pronounced regression of both types of tumors and in an increase of the subject life span.

Table 1 below illustrates different procedures which the composition of the present invention was prepared. The composition was used for testing sensitization of V-79 Chinese hamster and melanoma B-16 of mice cells to gamma-radiation by the addition of DN-1 to cell suspension.

TABLE 1

| | V-79 cells | | | |
|---|---|---|---|---|
| | Dose of | | B-16 cells | |
| | irradiation | | Dose of irradiation | |
| | resulting in a | | resulting in a | |
| | decrease | | decrease in cell | |
| Conditions of | in cell survival to | | survival to 20% | |
| irradiation | 20% of initial, Gy | DMF | of initial, Gy | DMF |
| Irradiation | 20 | 1 | 17 | 1 |
| without addition | | | | |
| of DN-1 | | | | |
| Irradiation in the | 14 | 1.4 | 12 | 1.4 |
| presence of DN-1 | | | | |
| Irradiation in the | 10 | 2.1 | 9 | 1.9 |
| presence of DN-1 | | | | |
| (II) | | | | |
| Irradiation in the | 11 | 2.0 | 9.5 | 1.8 |
| presence of DN-1 | | | | |
| (III) | | | | |

DN-1-homogenate of Halobacteria prepared on 7% NaCl; DN-1 (II)—homogenate with a double concentration of Halobacteria prepared on 7% NaCl.

DN-1 (III)—homogenate with a double concentration of Halobacteria prepared on 3.5% NaCl.

DMF a dose modification factor which presents how many times the irradiation dose may be reduced due to the presence of DN-1 in different preparations which result in the same decrease of cell survival as after irradiation in control (without DN-1).

FIG. 1 illustrates the survival of B-16 melanoma cells irradiated in vitro in suspension without DN-1 and with addition of 10% of DN-1 prepared in different ways:

DN-1—homogenate of Halobacteria prepared on 7% NaCl;

DN-1 (II)—homogenate with the double concentration of Halobacteria prepared on 7% NaCl.

DN-1 (III)—homogenate with the double concentration of Halobacteria prepared on 3.5% NaCl.

FIG. 2 illustrates a graph of dynamic of B-16 mouse melanoma growth after 25 Gy local gamma-irradiation.

Dynamic of B-16 mouse melanoma growth after 25 Gy local gamma-irradiation B-16 melanoma was transplanted in the calf muscle of the mouse.

The tumors were irradiated on the 9th day after transplantation when the calf muscle reached the mean diameter of 9 mm.

The mice were kept either on usual diet or received per os 0.2 ml daily of DN-1 homogenate prepared in one of three different procedures:

I-DN-1 prepared on 7% NaCl

II- Double concentration of DN-1 prepared on 7% NaCl

III-Reconstituted solution made from lyophilazed DN-1 prepared in a double concentration on 3.5% NaCl.

The above procedure was compared with mice which did not received DN-1 homogenate. Vt/Vo presents the ratio between Vt which is the mean tumor volume at the day of measurement, after treatment, and Vo which is the mean tumor volume at the day of irradiation.

FIG. 3-4 presents a graph of Dynamic of solid ELD carcinoma growth, transplanted into a mouse calf muscle, after exposure to 25 Gy local irradiation.

Ehrlich ELD carcinoma was transplanted in the calf muscle.

The tumors were irradiated on the 8th day after transplantation when the calfs reached the mean diameter of 8 mm.

The animals were kept either on usual diet or received per os 0.2 ml daily of 10% DN homogenate of Halobacteria prepared on 3.5% NaCl in one of three different procedures.

The graph presents:

▪ Animal group which did not receive DN;

[symbol] animal group which were fed with DN-1 prepared on 3.5% NaCl

Δ animal group which were fed with DN prepared on 3.5% NaCl with double concentration of Halobacteria

⋄ animal group which were fed with DN prepared on 3.5% NaCl with 4× concentration of Halobacteria

Vt/Vo presents the ration between Vt, the mean tumor volume at the day of measurement, after treatment, and Vo, the mean tumor volume at the day of irradiation.

FIG. 5 presents the survival percentage of mice with B-16 melanoma after local gamma irradiation (25 Gy) while animals were administered daily per os with 0.2 ml of 10% DN-1 or DN-1 (II) (III)—prepared with the double content of Halobacteria.

FIG. 6 presents survival of mice with ELD tumor after local gamma irradiation (25Gy) while animals were administered daily per os with 0.2 ml of 10% DN-1 or DN-1 (II) (III)—prepared with the double content of Halobacteria mass)

FIG. 7 Survival of V-79 cells irradiated in vitro in suspension without DN-1 and with addition of 10% of DN-1 prepared in different ways:

DN-1—homogenate of Halobacteria prepared on 7% NaCl;

DN-1 (II)—homogenate with the double concentration of Halobacteria prepared on 7% NaCl;

DN-1 (III)—homogenate with the double concentration of Halobacteria prepared on 3.5% NaCl.

Another example of the embodiments of the present invention done in-vitro will be presented.

The present invention discloses the formulated synergy extract of Dunaliella and Halobacteria, showing beneficial impacts on cancer cell lines platforms. The anti-proliferative and ability of Synergy to induce apoptosis in two types of cancer cell lines and to modulate their ability to invade in the Boyden chamber model in being disclosed.

Cancer Cell Lines Platform

Two types of certified cell lines were used: human skin sarcoma (obtained from CLS GmbH) and human skin melanoma (obtained from ATCC). We disclose the anti-cancer abilities of the extracts by examining their influences on cell proliferation (cytotoxicity) and apoptosis. In addition, invasion assay is disclosed to monitor cell movement through extracellular matrices (functioned in fundamental cellular processes such as angiogenesis, embryonic development, immune response, metastasis, and invasion of cancer cells).

Anti-Proliferative and Induction of Apoptosis

The assays were performed for each individual cell line. The cancer cells were seeded at 96 well plates in concentration of (approx.) 0.3×106 ¢/mL in 200 uL/well of fresh growth medium. The plates were incubated at 37° C. with 5% CO2 until reach 70% confluences (visual estimation). After the cells were reached the required confluences, the medium was replaced with pre-prepared growth medium stocks contain the maximal dose and the optimized ratio between Dunaliella and Halobacteria for the Synergy extract (200 uL/well). The plates were incubated overnight at 37° C. with 5% CO2. The viability and the extent of apoptosis in the cancer cells were measured after the incubation using MTT and Caspase 3 assays.

Invasion

The cancer cell lines were treated without or with the maximal non-lethal dose and the optimized ratio of Dunaliella and Halobacteria (20:80) for the Synergy extract. At the day of the assay, the required cells were mounted into the invasion chamber, according to the kit's instructions. The negative control was serum-free growth medium. The assay was carried out in triplicates. The invasive properties of the two types of tumor cells were determined following the kit protocol.

Results

Synergy (Dunaliella and Halobacteria with equal amount) reduced Melanoma and Sarcoma cells viability as shown in FIG. 8 and FIG. 9 respectively.

Combination of Dunaliella (20%) and Halobacteria (80%) synergistically reduce Melanoma and Sarcoma cell-line viability in a caspase-3 independent manner as shown in FIG. 10 and FIG. 11 respectively.

The cancer cell-lines were incubated without or with non-toxic levels of Dunaliella and Halobacteria for 24 hr. Then, cancer invasion through ECM-coated membrane was tested. Synergy blocked Sarcoma cell invasion, as shown in FIG. 12, but not Melanoma cell invasion, as shown in FIG. 13.

In conclusion, two major anti-cancer properties of the extracts were explored in this study. Initially, the extracts cytotoxicity in sarcoma- and melanoma cells was investigated. The results in FIGS. 8-9 indicate that Synergy exhibits specific cytotoxicity properties against Sarcoma cancer. In addition, Synergy had cytotoxic impact on the Melanoma cancel cells; however, it was similar to the effect of individual Halobacteria extract. At this concentration, no toxicity was detected at normal skin tissue (not shown). Next, the hypothesis that the extracts cytotoxicity was due to the induction of caspase 3-induced apoptosis was investigated. However, no significant effect was recorded in both cancer cell lines.

The invasion assay was performed to monitor movement through extracellular matrices, which is a fundamental cellular process in cancer metastasis. Therefore, the cancer cell lines were incubated without or with the extracts. The significant results of FIG. 12 clearly demonstrates that synergy blocked the ability of the Sarcoma cancer to migrate over the ECM-coated membrane.

Collectively, the results show the anti-sarcoma properties to the synergy compound.

Extraction Of Halobacteria Homogenate DN-1.

Homogenate from red halobacteria-Archea and microalgae Dunalielia isolated in 7.5% NaCl and pH =7

Extraction halobacteria homogenate DN-1 obtained from Halobacterium halobium, preparing a salt stock solution:

Adding 240L of NaCl, 30 L of MgCL₂*6H₂O, 35 L of MgSO₄*7H₂O, 7 L KCl to a flask. Adding pure water to near the final required volume of the same flask. The salt then dissolved using a magnetic stirrer.

Adding CaCl₂*2H₂O

Adjusting the pH of the flask solution up to 7 by using 1M TrisCl buffer.

Transferring the above solution to a graduated cylinder and toping up with water to the exact final volume.

A quantity of bacterial mass is dispersed beforehand and been added to the solution described below:

Adding 767 ml from the above salt stock solution, 200 ml of pure water, 5 g of peptone, 1 g of yeast extract, 3 g of microalgae powder, Dunaliella and 1 g of Casein.

Adjusting the volume with 1000 ml of pure water.

Sterilization the culture by using an autoclave.

Incubating the culture for sufficient time and temperature −37° C. yielding.

Suspending the culture for two weeks.

A solution of microalgae Dunaliella is added to a solution of 10-day halobacteria culture and the obtained mixture is incubated at temperature 25° C. and permanent illumination.

The algae is cultivated in a growth medium comprising:

| Salt | g/liter |
|---|---|
| 240 | NaCl |
| 30 | MgCL₂*6H₂O, |
| 35 | MgSO₄*7H₂O |
| 7 | KCl, |
| 0.2 | CaCl₂*2H₂O |
| 1.0 | KNO₃ |
| 0.035 | KH₂PO₄ |

The cultivation is adjusted to pH=7

Centrifugation of the 2 weeks old culture for 7500 RPM, 4° C., yielding a sediment Isolating the sediment and re-suspending it within a solution of 2M NaCl+0.15M MgCl₂.

Centrifugation of the solution is performed for 7500RPM, 4° C., yielding a sediment

Isolating the sediment layer and re-suspending it within a solution of 7.5% NaCl

Sonicating the above solution three times each time for 15sec. while in between each time cooling the solution in an ice bath until a different is shown in the color and turbidity or transparency of the solution

Centrifugation of the solution is performed yield separation fractions (the centrifugation procedure is performed in 7500 RPM 4° C. for 10 min) The resulting sediment is isolated and kept in (−4)° C.

Method of Oral Tablet Preparation

The base ingredient listed below was mixed for about 20 minutes in a Day powder mixer or a Pony mixer. The extract was added to the base mixture and the mixture was again mixed for about 30 minutes adding lubricants. Finally the complete mixture was permitted to temper for bonding for not less than 24 hours at room temperature. The tablets were compressed into scored capsule-shaped oral tablets.

Dispensing the weight of each ingredient in the mixture is determined according to determined dose. Sizing a fine particle size for greater uniformity of dose (size reduction, milling, crushing, grinding, pulverization).

Granulation following particle size reduction and blending, the formulation may be granulated, which provides homogeneity of drug distribution in blend.

Drying by keeping the residual moisture low enough to prevent product deterioration and ensure free flowing properties. (The drying process may preformed using a Fluidized-bed dryer, Vacuum tray dryer, Microwave dryer, Spray dryer, Freeze dryer, Turbo-tray dryer, or Pan dryer).

Tablet compression is performed after the preparation of granules (in case of wet granulation) or sized slugs (in case of dry granulation) or mixing of ingredients (in case of direct compression), they are compressed to get final product

In the foregoing description, embodiments of the invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

What is claimed is:

1. A composition for treating solid tumors in a mammalian subject said composition comprising halobacterial extracts containing antioxidants comprising:
a. at least one water soluble fraction; and
b. at least one oil soluble fraction
wherein said halobacterial extract is adapted for therapeutic treatment of solid tumor reduction; further wherein said halobacterial extract promotes solid tumor reduction in radiation treatment.

2. The composition according to claim 1, wherein said composition further comprises Dunaliella extracts.

3. The composition according to claim 1, wherein said halobacteria extract is in an amount by weight of about 2.5%-10%.

4. The composition according to claim 1, wherein said halobacterial extract comprises a therapeutic dose of halobacteria homogenate DN-1 (halophilic archaea homogenate).

5. The composition according to claim 1, wherein said antioxidants composition confers a synergistic therapeutic radiosensitivity effect on subjects with respect to reduction of solid tumor.

6. The composition according to claim 5, wherein said synergistic effect is defined:
a. as at least 50% higher radiosensitivity to radiation treatment on cancer cells with respect to increasing the radiation damage of tumor cells in vitro;
b. as a higher radiosensitivity to radiation treatment on subjects with respect to more pronounced regression of tumors; and
c. as an increscent of life span in comparison to a control group;

7. The composition according to claim 1, wherein said composition is administered orally and said orally administered composition is in a form selected from the group consisting of: tablets, capsules, caplets, or any oral suitable form.

8. The composition according to claim 1, wherein said Halobacteria extract:
a. DN-1(I)—homogenate, prepared on 7% NaCl results a regression of about 10% tumor cells and results less than 10% survival of tumor cells after irradiation of more than 16 Gy;
b. DN-1 (II) homogenate, with the double concentration of Halobacteria prepared on 7% NaCl results a regression of about 10% tumor cells and results less than 10% survival of tumor cells after irradiation of more than 10 Gy; and
c. DN-1(III)—homogenate, with the double concentration of Halobacteria prepared on 3.5% NaCl results a regression of about 10% tumor cells and results less than 10% a survival of tumer cells after irradiation of more than 10 Gy.

9. The composition according to claim 1, wherein said Halobacteria extract, halobacteria homogenate DN-1:
a. has a modifying effect on radiosensitivity with gamma irradiation at 5 and 10 Gy;
b. effect upon the radiosensitivity of tumor cells irradiated in suspension results in pronounced regression of tumor cells and in an increscent of a mammalian life span;
c. increase the radiation damage of tumor cells in vitro and in vivo;
d. reduces tumor cell proliferation; and
e. results the effect of suppression of malignant cell growth; where said effect increases proportionally to DN-1 concentration and to the duration of treatment.

10. A solid tumor reduction composition comprising halobacterial, wherein said composition:
a. is characterized by at least one measurable biological activity as determined by at least one bioassay, further wherein said composition has a anti tumorogenic activity fingerprint defined by Table 1; and
b. acts as an anti-angiogenesis factor and is characterized by interrupting the process of angiogenesis of solid tumor by facilitating a process selected from the group consisting of inhibiting proteases, inhibiting integrin signaling, inhibiting binding and activity of VEGF, affecting cell proliferation of endothelial cells, inhibiting bFGF, inhibiting cell migration, inducing apoptosis of endothelial cells, cell adhesion and survival of endothelial cells, antagonist of angiopoietin 1, or decoy receptors for VEGF-B and PIGF.

11. An oral administrable tablet useful for treating irradiation and other related disorders, said tablet comprising: a halobacterial extract fraction, a Dunaliella extract fraction, at least one non-effervescent excipient or diluent, and at least one effervescent excipient.

12. A method for extracting halobacteria homogenate DN-1, comprising the steps of:
a. providing halophilic bacteria, the bacteria being cultured in a medium which prevents rupture of the bacteria;
b. surrounding the bacteria in a medium comprising a bile salt and having a concentration of NaCl low enough to lyse the bacteria and cause said halobacteria homogenate DN-1 to be released into the medium; and
c. isolating the released halobacteria homogenate DN-1;
wherein said halobacteria homogenate DN-1 is adapted for therapeutic treatment of solid tumor reduction; further wherein said halobacteria homogenate DN-1 promotes solid tumor reduction in radiation treatment.

13. The method according to claim 12, wherein the medium in step (b) further comprises a cation complexing agent and a detergent selected from the group consisting of anionic detergents, cationic detergents, nonionic detergents, and amphoteric detergents.

14. The method according to claim 12, wherein said halobacteria extract:
a. is in an amount by weight of about 2.5%-10%; and
b. comprises a therapeutic dose of halobacteria homogenate DN-1.

15. The method according to claim 12, wherein said antioxidants composition confers a synergistic therapeutic radio sensitivity effect on subjects with respect to reduction of solid tumor.

16. The method according to claim 15, wherein said synergistic effect is:
a. at least 50% higher radiosensitivity to radiation treatment on cancer cells with respect to increasing the radiation damage of tumor cells in vitro;
b. defined as a higher radiosensitivity to radiation treatment on subjects with respect to more pronounced regression of tumors;
c. defined as an increase of life span in comparison to a control group.

17. The method according to claim 12, wherein said composition is administered orally and said orally administered composition is in the form selected from the group consisting of: tablets, capsules, caplets, or any oral suitable form.

18. The method according to claim 12, wherein said Halobacteria extract, halobacteria homogenate DN-1, has a modifying effect on radiosensitivity with gamma irradiation at 5 and 10 Gy.

19. The method according to claim 12, wherein said Halobacteria extract:
a. halobacteria homogenate DN-1(I), prepared on 7% NaCl results a regression of about 10% tumor cells and less than 10% survival of tumor cells after irradiation of more than 16 Gy;
b. DN-1 (II) homogenate, with the double concentration of Halobacteria prepared on 7% NaCl results a regression of 10% tumor cells and less than 10% survival of tumor cells after irradiation of more than 10 Gy; and
c. DN-1(III)—homogenate, with the double concentration of Halobacteria prepared on 3,5% NaCl results a regression of about 10% tumor cells and less than 10% a survival of tumer cells after irradiation of more than 10 Gy.

20. The method according to claim 12, wherein said Halobacteria extract, DN-1 Halobacteria homogenate:
a. effect upon the radiosensitivity of tumor cells irradiated in suspension results in:
i. pronounced regression of tumor cells;
ii. an increase of a mammalian life span.
b. increases the radiation damage of tumor cells in vitro and in vivo;
c. reduces tumor cell proliferation;
d. results in the effect of suppression of malignant cell growth and said effect increases proportionally to DN-1 concentration and to the duration of treatment.
